(19) United States
(12) Patent Application Publication
(10) Pub. No.: US 2025/0302278 A1
(43) Pub. Date: Oct. 2, 2025

(54) ENDOSCOPE SYSTEM, CONTROL DEVICE, CONTROL METHOD, AND STORAGE MEDIUM

(71) Applicants: OLYMPUS CORPORATION, Tokyo (JP); NATIONAL CANCER CENTER, Tokyo (JP)

(72) Inventors: Masafumi HARAGUCHI, Tokyo (JP); Naoya HATAKEYAMA, Tokyo (JP); Masaaki ITO, Tokyo (JP); Hiro HASEGAWA, Tokyo (JP); Daichi KITAGUCHI, Tokyo (JP)

(73) Assignees: OLYMPUS CORPORATION, Tokyo (JP); NATIONAL CANCER CENTER, Tokyo (JP)

(21) Appl. No.: 19/049,859

(22) Filed: Feb. 10, 2025

(30) Foreign Application Priority Data
Mar. 28, 2024 (JP) 2024-052782

(51) Int. Cl. A61B 1/00

(52) U.S. Cl. CPC A61B 1/00045; A61B 1/00006; A61B 1/00147; A61B 1/00179; A61B 1/00188

(57) ABSTRACT

An endoscope system includes: a processor; an endoscope; a holder configured to hold the endoscope and adjust a position and an orientation of the endoscope; and a display. The processor acquires a first position of a first port for inserting, into an examination subject, a first treatment tool, a second position of a second port for inserting, into the examination subject, a second treatment tool, and a third position of a focus target in the examination subject, and rotates the image on a basis of the acquired first, second, and third positions such that a particular relative angle is formed between a direction of a second vector, which is a projection of a first vector indicating an up-down direction of the focus target as viewed from an operator onto a plane orthogonal to a visual axis of the endoscope and an up-down direction of the image displayed on the display.

### CROSS-REFERENCE TO RELATED APPLICATIONS

This application is based on Japanese Patent Application No. 2024-052782, the content of which is incorporated herein by reference.

### TECHNICAL FIELD

The present disclosure relates to an endoscope system, a control device, a control method, and a storage medium.

### BACKGROUND

There is known a surgical robot that controls the rotation angle of an insertion portion of a surgical endoscope about the longitudinal axis.

### SUMMARY

An aspect of the present disclosure is an endoscope system comprising: at least one processor comprising hardware; an endoscope; a holder that is configured to hold the endoscope and adjust a position and an orientation of the endoscope by a control signal from the at least one processor; and a display that displays an image acquired by the endoscope, wherein the at least one processor is configured to: acquire a first position of a first port for inserting, into an examination subject, a first treatment tool operated by an operator, a second position of a second port for inserting, into the examination subject, a second treatment tool operated by the operator, and a third position of a focus target in the examination subject, and rotate the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

Another aspect of the present disclosure is an endoscope system comprising: at least one processor; an endoscope; a moving device that holds the endoscope and adjusts a position and an orientation of the endoscope by a control signal from the processor; and a display device that displays an image acquired by the endoscope, in which the processor acquires a first position of a first port for inserting, into an examination subject, a first treatment tool operated by an operator, a second position of a second port for inserting, into the examination subject, a second treatment tool operated by the operator, and a third position of a focus target in the examination subject, and controls the moving device and/or the endoscope so that the visual axis of the endoscope comes closer to a straight line that connects the third position and a fourth position between the first position and the second position.

Another aspect of the present disclosure is a control device of an endoscope system comprising an endoscope, a holder that is configured to hold the endoscope and adjust a position and an orientation of the endoscope, and a display that displays an image acquired by the endoscope, the control device comprising: at least one processor comprising hardware, wherein the at least one processor is configured to: acquire a first position of a first port for inserting, into an examination subject, a first treatment tool operated by an operator, a second position of a second port for inserting, into the examination subject, a second treatment tool operated by the operator, and a third position of a focus target in the examination subject, and rotate the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

Yet another aspect of the present disclosure is a control method of an endoscope system comprising an endoscope, a holder that is configured to hold the endoscope and adjust a position and an orientation of the endoscope, and a display that displays an image acquired by the endoscope, the control method comprising: acquiring a first position of a first port for inserting, into an examination subject, a first treatment tool operated by an operator, a second position of a second port for inserting, into the examination subject, a second treatment tool operated by the operator, and a third position of a focus target in the examination subject; and rotating the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

Still another aspect of the present disclosure is a non-transitory computer-readable storage medium storing a program for controlling an endoscope system comprising an endoscope, a holder that is configured to hold the endoscope and adjust a position and an orientation of the endoscope, and a display that displays an image acquired by the endoscope, the program comprising instructing a computer to execute: acquiring a first position of a first port for inserting, into an examination subject, a first treatment tool operated by an operator, a second position of a second port for inserting, into the examination subject, a second treatment tool operated by the operator, and a third position of a focus target in the examination subject; and rotating the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

### BRIEF DESCRIPTION OF DRAWINGS

FIG. 1 is a general structural diagram of an endoscope system according to one embodiment of the present disclosure.

FIG. 2 is a block diagram illustrating the endoscope system illustrated in FIG. 1.

FIG. 3 is a schematic diagram illustrating laparoscopic surgery on a patient by using the endoscope system illustrated in FIG. 1.

FIG. 4 is a partial vertical sectional view of an endoscope and a trocar of the endoscope system illustrated in FIG. 1.

FIG. 5 is a flowchart illustrating a control method of the endoscope system illustrated in FIG. 1.

FIG. 6 is a schematic diagram illustrating a control method of the endoscope system illustrated in FIG. 5.

FIG. 7A is a diagram in which a second vector and an endoscope image when the up-down direction of an imaging element of the endoscope aligns with the vertical direction are superimposed.

FIG. 7B is a diagram in which the endoscope image in FIG. 7A is rotated so that the up-down direction of the imaging element of the endoscope is coincident with the second vector.

FIG. 8 is a schematic diagram illustrating the case where the focus target is switched in the endoscope system illustrated in FIG. 1.

FIG. 9A is a diagram in which the second vector and an endoscope image that images a focus target at a position closer to the operator than the case illustrated in FIG. 7A and that has the up-down direction of the imaging element of the endoscope aligned in the vertical direction are superimposed.

FIG. 9B is a diagram in which the endoscope image in FIG. 9A is rotated so that the up-down direction of the imaging element of the endoscope is coincident with the second vector.

FIG. 10 is a flowchart illustrating a modification of the control method illustrated in FIG. 5.

FIG. 11 is a flowchart illustrating another modification of the control method illustrated in FIG. 5.

FIG. 12 is a diagram illustrating a modification of the endoscope system illustrated in FIG. 1.

FIG. 13 is a diagram illustrating a modification of the endoscope system illustrated in FIG. 1, which is an endoscope system that includes an oblique-viewing endoscope.

FIG. 14 is a diagram illustrating a modification of the endoscope system illustrated in FIG. 1, which is an endoscope system that includes an endoscope having a bending portion.

### DESCRIPTION OF EMBODIMENTS

An endoscope system 1, a control device 5, a control method, and a program according to one embodiment of the present disclosure will now be described with reference to the drawings.

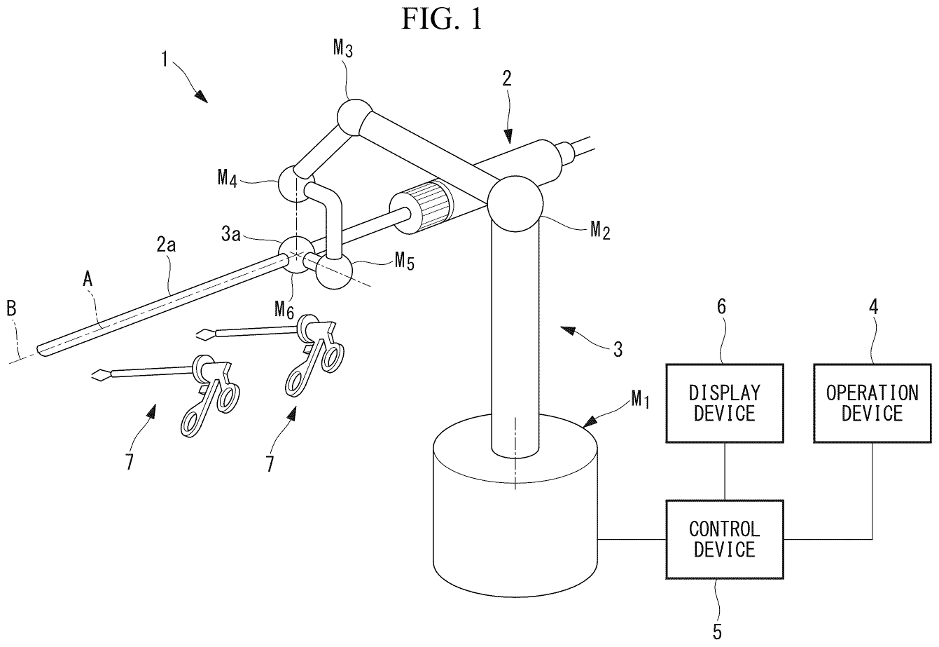

As illustrated in FIG. 1, the endoscope system 1 according to an embodiment is used in surgery, such as laparoscopic surgery, that involves inserting an endoscope 2 and treatment tools 7 into the body of a patient (examination subject) D and treating the target site such as an affected area with the treatment tools 7 while observing the treatment tools 7 through the endoscope 2.

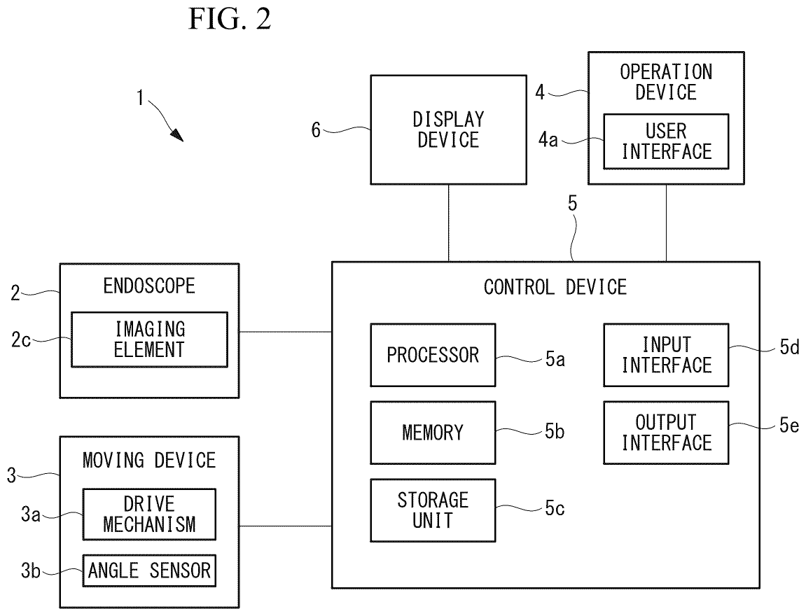

As illustrated in FIGS. 1 and 2, the endoscope system 1 includes the endoscope 2, a moving device 3 that holds and moves the endoscope 2, an operation device 4 operated by the user, a control device 5 that controls the moving device 3 on the basis of an operation signal from the operation device 4, and a display device 6. The endoscope 2 includes a tubular scope barrel portion (insertion portion) 2a extending along a longitudinal axis A and is a direct viewing rigid scope having a visual axis B coincident with the longitudinal axis A.

The endoscope 2 includes an imaging element 2c that is inserted into the body of the patient D along with one or more treatment tools 7 and that acquires an endoscope image (image) C including one or more treatment tools 7. The endoscope 2 sends the endoscope image C acquired by the imaging element 2c to the display device 6 via the control device 5. The imaging element 2c is, for example, a three-dimensional camera installed at a proximal end of the endoscope 2, and acquires a stereo image as the endoscope image C. The display device 6 is any desired display such as a liquid crystal display or an organic EL display. The operator E operates the treatment tools 7 while observing the endoscope image C displayed on the display device 6.

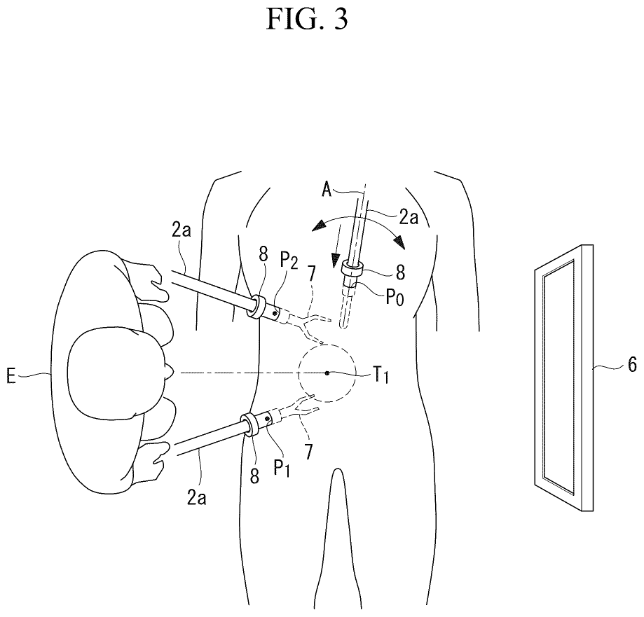

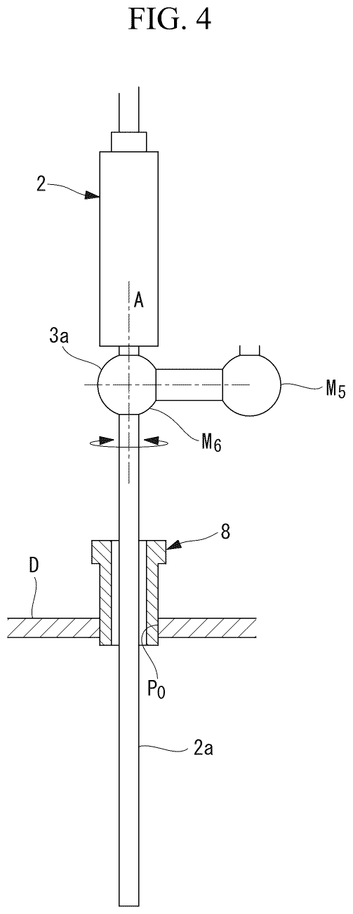

As illustrated in FIGS. 3 and 4, the endoscope 2 and the treatment tools 7 are inserted into the body, for example, the abdominal cavity, of the patient D via respective trocars 8. The trocars 8 are tubular tools attached to holes P₀, P₁, and P₂ in the abdominal wall, and are pivotable about pivot points near the holes P₀, P₁, and P₂.

The moving device 3 is an articulated robot arm having at least six joints M₁ to M₆. The moving device 3 includes a drive mechanism 3a that serves as the joint M₆ at the tip holding the endoscope 2 and that drives and rotates the endoscope 2 about the longitudinal axis A. The position and the orientation of the endoscope 2 are three-dimensionally adjusted by the movement of the moving device 3. The moving device 3 includes an angle sensor 3b that detects the angle of each of the joints M₁ to M₆. The angle sensor 3b is, for example, an encoder, a potentiometer, or a Hall sensor installed in each of the joints M₁ to M₆.

As illustrated in FIG. 2, the control device 5 includes at least one processor 5a, at least one memory 5b, a storage unit (memory) 5c, an input interface 5d, and an output interface 5e. The control device 5 controls the endoscope 2, the moving device 3, and the endoscope image C displayed on the display device 6. Furthermore, the control device 5 is connected to the endoscope 2, the moving device 3, the operation device 4, and the display device 6 via the input interface 5d and the output interface 5e, and sends and receives the endoscope image C, signals, etc., via the input interface 5d and the output interface 5e.

The storage unit 5c is a computer-readable, non-transitory storage medium, and examples thereof include a hard disk drive, an optical disk, and a flash memory. The storage unit 5c stores a control program that instructs the processor 5a to execute a control method described below, and data necessary for the processing by the processor 5a. In addition, the storage unit 5c stores link lengths, which are the lengths of the links between the joints M₁ to M₆ of the moving device 3.

The processor 5a executes a control method described below according to the control program read into the memory 5b, such as a random access memory (RAN), from the storage unit 5c. Some of the processes described below executed by the processor 5a may be realized by a dedicated logic circuit, hardware, or the like such as a field programmable gate array (FPGA), a system-on-a-chip (SoC), an application specific integrated circuit (ASIC), or a programmable logic device (PLD).

The processor 5a may control the moving device 3 according to a follow mode or a stay mode. The user such as the operator can select one of the stay mode and the follow mode by using a user interface (not illustrated) installed in the control device 5.

The stay mode is a mode in which the endoscope 2 stays at a particular position irrespective of the positions of the treatment tools 7.

The follow mode is a mode in which the processor 5a controls the moving device 3 on the basis of the positions of the treatment tools 7 so that the endoscope 2 automatically follows the treatment tools 7. For example, the processor 5a acquires, by stereo measurement using the endoscope image C, the positions of the tips of the treatment tools 7, and controls the moving device 3 to move the endoscope 2 such that the tips of the treatment tools 7 are within the endoscope image C.

Typically, the moving device 3 changes the position and the orientation of the endoscope 2 while maintaining the up-down direction of the imaging element 2c so that the up-down direction of the endoscope image C acquired by the endoscope 2 remains unchanged.

In the endoscope system 1 of this embodiment, the moving device 3 changes the position and the orientation of the endoscope 2 by rotating the endoscope 2 about its longitudinal axis A according to the control method described below.

Next, the method for controlling the endoscope system 1 according to the embodiment is described with reference to the drawings.

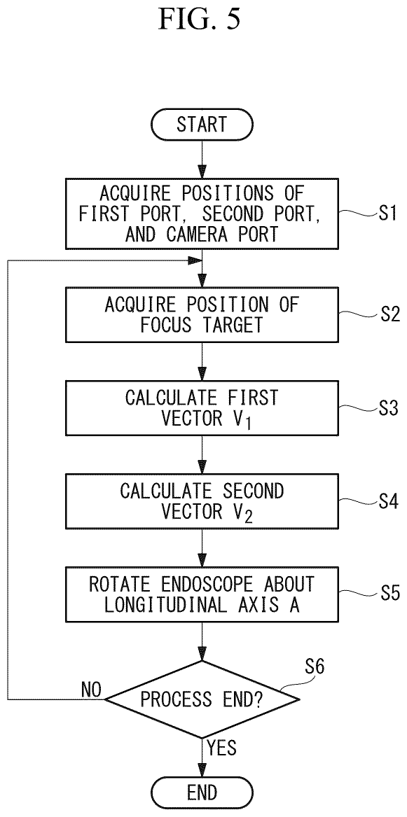

As illustrated in FIG. 5, the processor 5a acquires the three-dimensional positions of the holes (ports) P₀, P₁, and P₂ in the abdominal wall for attaching the trocars 8 (step S1).

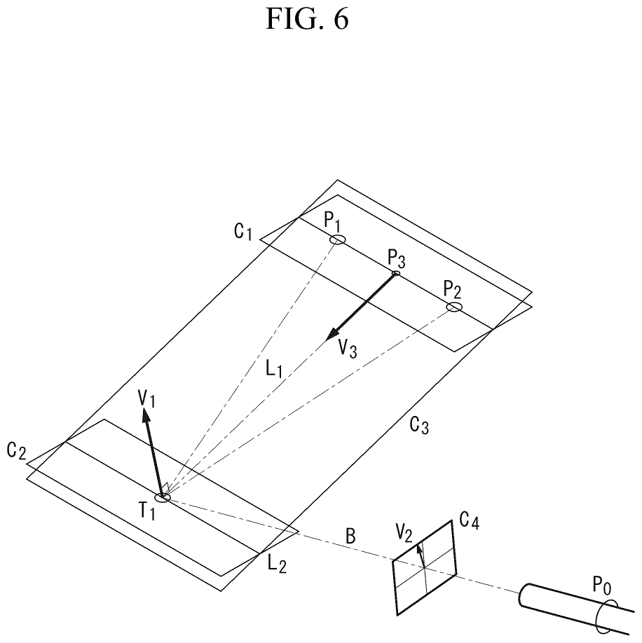

The trocars 8 are respectively attached to a camera port P₀ for inserting the endoscope 2, and first and second ports P₁ and P₂ for inserting two treatment tools 7. The first port P₁ is a hole to which the trocar 8 for inserting the treatment tool (first treatment tool) 7 operated by the right hand of the operator E is attached. The second port P₂ is a hole to which the trocar 8 for inserting the treatment tool (second treatment tool) 7 operated by the left hand of the operator E is attached. In FIG. 6, a plane C₁ represents the abdominal wall, in which the first port P₁ and the second port P₂ lie, schematically illustrated as a horizontal plane for the sake of convenience.

The three-dimensional position of the camera port P₀ and the three-dimensional positions of the first and second ports P₁ and P₂ (first and second position) are acquired by measurement or by numeric input. Each of the three-dimensional positions may be acquired by, for example, actuating the moving device 3 having a probe (not illustrated) at the tip, bringing the probe into contact with each of the ports P₀, P₁, and P₂. and acquiring the three-dimensional positions from the angle information at the joints M₁ to M₆ of the moving device 3 acquired by the angle sensor 3b.

Next, the processor 5a acquires the three-dimensional position (third position) of a focus target T₁ of an examination subject in the abdominal cavity (step S2). The focus target T₁ is a tip point of one of the treatment tools 7 in the endoscope image C acquired by the endoscope 2 or the point at the image center of the endoscope image C, for example. The three-dimensional position of the focus target T₁ is calculated on the basis of the link lengths and the angles of the joints M₁ to M₆ of the moving device 3 in a state where the endoscope 2 is inserted through the camera port P₀ and the distance to the focus target T₁ calculated from the endoscope image C. In FIG. 6, a plane C₂ represents an image subject, in which the focus target T₁ lies, schematically illustrated as a horizontal plane for the sake of convenience.

Then the processor 5a calculates a first vector V₁ indicating the up-down direction of the focus target T₁ as seen from the operator E on the basis of the acquired first, second, and third positions (step S3). As illustrated in FIG. 6, the first vector V₁ is defined in a direction orthogonal to a plane C₃ that starts from the third position and includes the first, second, and third positions. The magnitude of the first vector V₁ may be any.

A third vector V₃ that indicates the direction of the line of sight of the operator E is defined to be on a straight line L₁ that connects the focus target T₁ and a point between the first and second ports P₁ and P₂ on the aforementioned plane C₃, for example, the center position (fourth position) P₃. In other words, the presumptions for the setting are that the head H of the operator E is positioned between the treatment tool 7 held by the right hand and the treatment tool 7 held by the left hand and that the height of the eyes is approximately at the extension of the plane C₃. The magnitude of the third vector V₃ may also be any.

The processor 5a then calculates the direction of a second vector V₂ which is a projection of the first vector V₁ onto a plane C₄ orthogonal to the visual axis B of the endoscope 2 (step S4). The visual axis B of the endoscope 2 is defined to be on a straight line L₂ that connects the three-dimensional position of the camera port P₀ and the three-dimensional position of the focus target T₁.

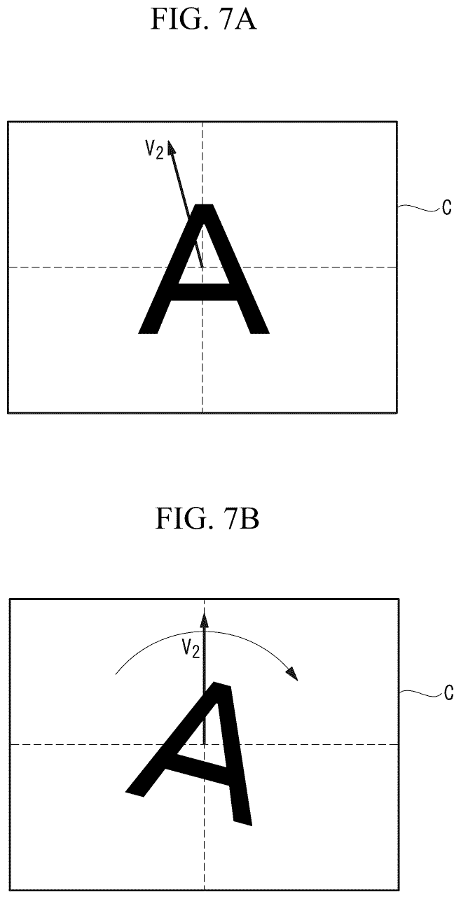

FIG. 7A is a diagram in which the second vector V₂ and the endoscope image C acquired while the up-down direction of the imaging element 2c is maintained in the vertical direction are superimposed. In the drawing, “A” schematically represents the image subject in the abdominal cavity. The second vector V₂ does not have to be displayed on the display device 6.

The third vector V₃ indicating the direction of the line of sight of the operator E extends along the straight line L₁ that connects the center position P₃ on the abdominal wall and the focus target T₁ in the body, and is slanted with respect to the planes C₁ and C₂, which are horizontal planes. Thus, the first vector V₁ is slanted with respect to the vertical direction when viewed from directions other than the direction along the straight line L₁, and the second vector V₂ projected onto the plane C₄ orthogonal to the straight line L₂ along the visual axis B not parallel to the straight line L₁ is also slanted with respect to the vertical direction.

The processor 5a sends a control signal to the drive mechanism 3a, and rotates the endoscope 2 about the longitudinal axis A so that the up-down direction of the endoscope image C displayed on the display device 6 is coincident with (having a relative angle of 0°) the direction of the second vector V₂ (step S5). As a result, as illustrated in FIG. 7B, the endoscope image C displayed on the display device 6 rotates in the arrow direction about the image center, and the second vector V₂ points vertically upward.

Then the processor 5a judges whether to end the process or not (step S6), and if NO, the steps from step S2 are repeated.

As such, according to the endoscope system 1, the control device 5, the control method, and the program of this embodiment, the endoscope image C is rotated so that the direction of the second vector V₂ is coincident with the up-down direction of the endoscope image C displayed on the display device 6.

Thus, it becomes possible to align the up-down direction of the endoscope image C and the up-down direction for the operator E operating the treatment tool 7 while observing the endoscope image C on the display device 6. In this manner, the operator E can intuitively operate the treatment tool 7. For example, the operator E can move the treatment tool 7 up or down also in the endoscope image C displayed on the display device 6 by intuitively operating and moving the tip of the treatment tool 7 straight up or down.

In addition, in the follow mode, when the treatment tool 7 has been moved, the processor 5a controls the moving device 3 to move the endoscope 2 such that the tip of the treatment tool 7 comes at, for example, the image center of the endoscope image C. Since this changes the first vector V₁ and the second vector V₂, the processor 5a actuates the drive mechanism 3a so that the up-down direction of the endoscope image C displayed on the display device 6 is always coincident with the direction of the second vector V₂.

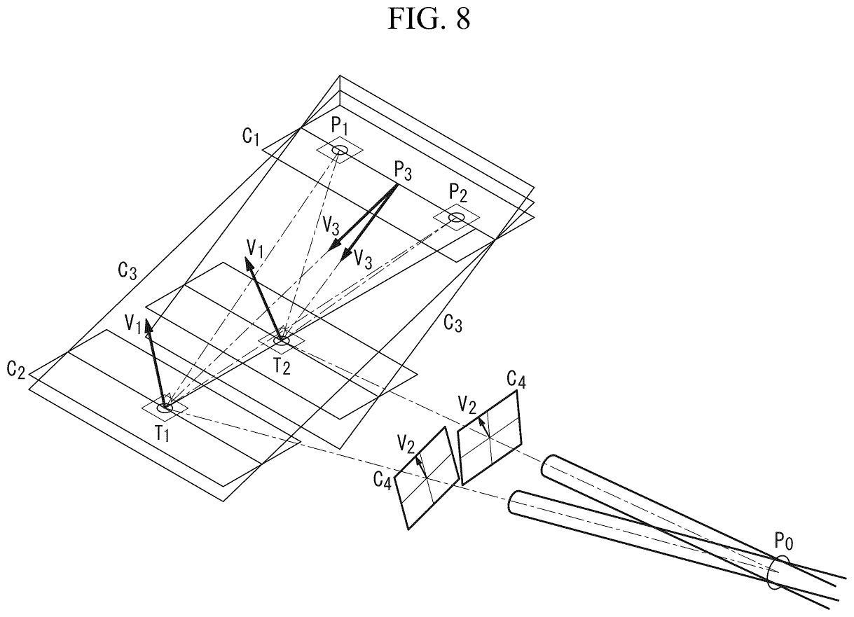

For example, as illustrated in FIG. 8, the case in which the tip of the treatment tool 7 is moved from a focus target T₁ which is at a relatively far position in the horizontal direction from the first port P₁ and the second port P₂, to a focus target T₂ which is at a relatively close position is described. In an endoscope image C imaging the focus target T₁, as illustrated in FIG. 8, the angles formed by the plane C₃ with respect to the horizontal planes C₁ and C₂ are relatively small, and thus the angle formed by the second vector V₂ with respect to the up-down direction of the acquired endoscope image C is relatively small. In contrast, in an endoscope image C imaging the focus target T₂ disposed at a relatively close position in the horizontal direction from the first port P₁ and the second port P₂, the angles formed by the plane C₃ with respect to the planes C₁ and C₂ are relatively large. Thus, the angle formed by the second vector V₂ with respect to the up-down direction of the acquired endoscope image C is relatively large.

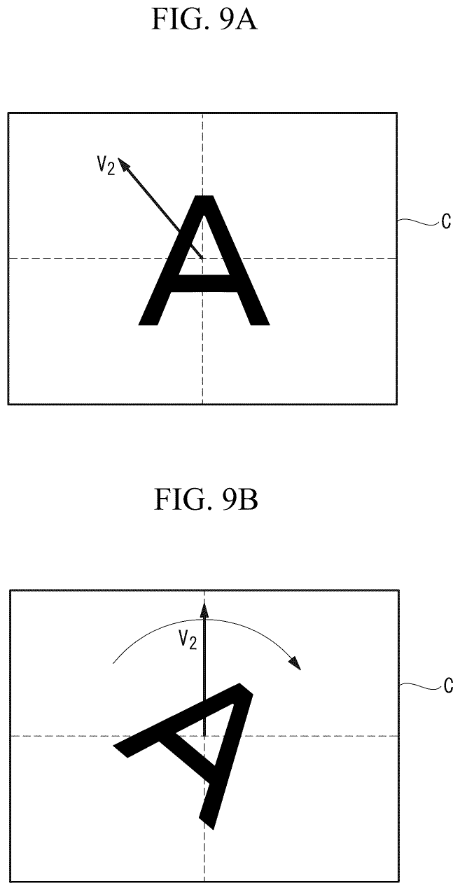

In other words, as illustrated in FIGS. 7A and 9A, if the endoscope image C acquired while maintaining the up-down direction of the imaging element 2c in the vertical direction is directly displayed on the display device 6, the up-down direction recognized by the operator E with respect to the up-down direction of the endoscope image C is different depending on whether the focus target is T₁ or T₂. This renders it difficult for the operator E to accurately operate the treatment tools 7. According to this embodiment, as illustrated in FIGS. 7B and 9B, the up-down direction of the endoscope image C displayed on the display device 6 is always coincident with the direction of the second vector V₂, and this provides an advantage in that the operator E can always accurately and intuitively operate the treatment tools 7 irrespective of the switch of the focus targets T₁ and T₂.

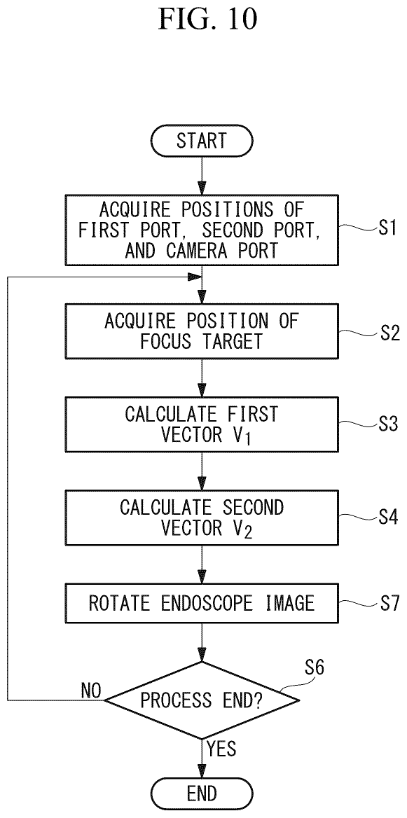

Note that, in this embodiment, the processor 5a actuates the drive mechanism 3a of the moving device 3 to rotate the endoscope 2 about the longitudinal axis A so that the angle of view of the endoscope image C acquired by the imaging element 2c is rotated (step S5). Alternatively, as illustrated in FIG. 10, the processor 5a may perform image processing on the endoscope image C acquired by the imaging element 2c so as to rotate the endoscope image C about the image center (step S7). The advantage provided is that, since the endoscope 2 is not physically rotated, there is no need to install the drive mechanism 3a in the moving device 3, and the structure of the endoscope system 1 can be simplified.

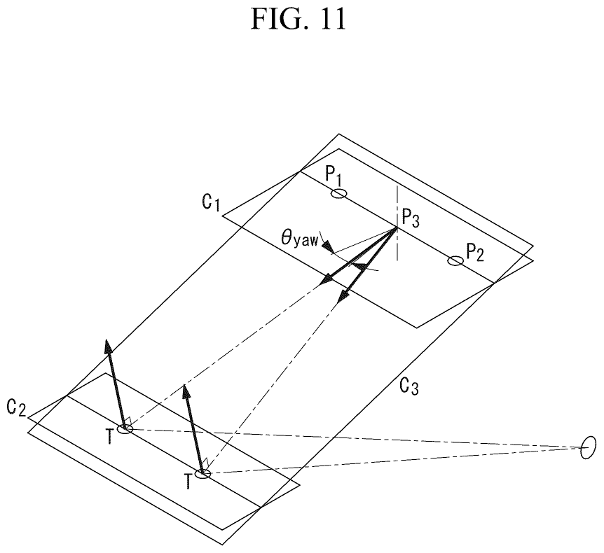

In addition, in this embodiment, the first vector V₁ orthogonal to the plane C₃ that includes the positions of the first port P₁, the second port P₂ and the focus target T₁ is defined, and the rotation angle of the endoscope image C is calculated on the basis of the second vector V₂ which is a projection of the first vector V₁ onto the plane C₄ orthogonal to the longitudinal axis A of the endoscope 2. Alternatively, as illustrated in FIG. 11, when the third vector V₃ indicating the direction of the line of sight of the operator E rotates about the vertical axis by the switch of the focus targets T₁ and T₂, the endoscope image C may be rotated by a rotation angle θᵣₒₗₗ determined by the function f(θ_{yaw}) of the rotation angle θ_{yaw}.

Specifically, the processor 5a defines the third vector V₃ in the direction of the line of sight of the operator E, and calculates the rotation angle θ_{yaw} of a vector, which is a projection of the third vector V₃ onto the horizontal plane C₁, about the vertical axis generated by the switch of the focus targets T₁ and T₂.

Then, for example, the angle θᵣₒₗₗ is calculated from the following equation:

$${θ}_{roll} = f ( {θ}_{yaw} ) = K {θ}_{yaw}$$

Here, K represents a constant.

In this manner, calculation of the first vector V₁ and the second vector V₂ is no longer necessary, and the rotation angle θᵣₒₗₗ of the endoscope image C can be determined by a simple computation; thus, there is an advantage in that the amount of calculation can be decreased.

Moreover, in this embodiment, the endoscope image C is rotated so that the second vector V₂ is coincident with the up-down direction of the endoscope image C displayed on the display device 6. Alternatively, the endoscope image C may be rotated so that the second vector V₂ forms a particular angle with respect to the up-down direction of the endoscope image C displayed on the display device 6. Some operator E may find it easier to operate the treatment tools 7 when the direction of the second vector V₂ is not coincident with the up-down direction of the endoscope image C; thus, the arrangement may be made to set the angle preferred by the operator E to one or both of a preset value and an offset value.

In addition, the positions of the first port P₁, the second port P₂, and the camera port P₀ may be stored in advance in the storage unit 5c in association with the type of procedure to be carried out. The processor 5a may read out and acquire the associated position information from the storage unit 5c by letting the user, including the operator E, input the type of the procedure to be carried out via the user interface 4a.

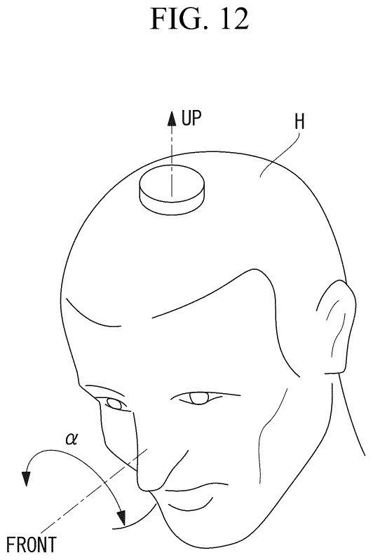

Furthermore, as illustrated in FIG. 12, the endoscope system 1 may include a sensor 9, such as a gyro sensor or a position sensor, attached to the head H of the operator E, and the processor 5a may adjust the slope of the first vector V₁ on the basis of the slope of the head H detected with the sensor 9. The up-down direction recognized by the operator E changes depending on the slope a of the head H in the left-right direction; thus, the first vector V₁ may be calculated by taking the slope a into account. In this manner, the up-down direction of the endoscope image C displayed on the display device 6 can coincide with the up-down direction recognized by the operator E even when the operator E tilts the head H during the procedure.

In this embodiment, a direct viewing rigid scope having the visual axis B coincident with the longitudinal axis A direction of the insertion portion 2a is described as an example of the endoscope 2. Alternatively, an oblique-viewing endoscope 2 that has a visual axis B slanted with respect to the longitudinal axis A or an endoscope 2 that has, at the tip of the insertion portion 2a, a bending portion 2b that can change the direction of the visual axis B may be employed. In such a case, the processor 5a may control the moving device 3 and/or the endoscope 2 so that the direction of the visual axis B shifts closer to the direction of the third vector V₃.

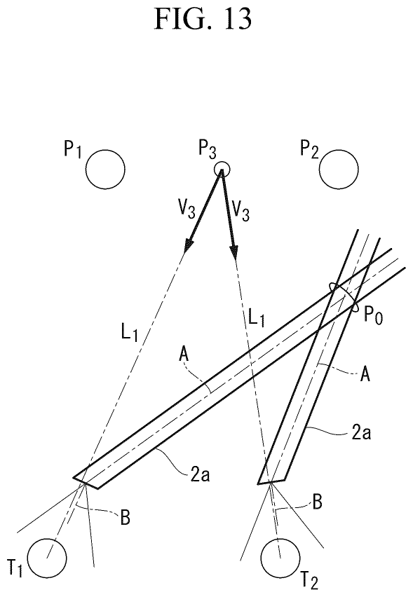

In other words, the case in which the endoscope 2 is of an oblique-viewing type is described with reference to FIG. 13. When the endoscope 2 is of an oblique-viewing type, the endoscope 2 may be inserted into the body in a direction different from the straight line L₁, and yet the focus targets T₁ and T₂ can be placed within the field of view while the tip of the endoscope 2 is placed on the straight line L₁.

The processor 5a controls the moving device 3 so that the visual axis B is approximately parallel to the straight line L₁ and that the distance is smaller than a predetermined threshold. In particular, the moving device 3 is preferably controlled such that the visual axis B is coincident with the straight line L₁. In this manner, the up-down direction of the endoscope image C acquired by the imaging element 2c approximately coincides with the up-down direction recognized by the operator E, and thus the process of rotating the endoscope image C is not necessary. In this manner also, the operator E can intuitively operate the treatment tool 7. Here, when the visual axis B and the straight line L₁ are not completely coincident with each other after the moving device 3 is controlled to make the visual axis B coincident with the straight line L₁, the endoscope image C may be rotated on the basis of the acquired first, second, and third positions such that a particular relative angle is formed between the up-down direction of the endoscope image C displayed on the display device 6 and the direction of the second vector V₂, which is a projection of the first vector V₁ indicating the up-down direction of the focus target viewed from the operator E onto a plane orthogonal to the visual axis B.

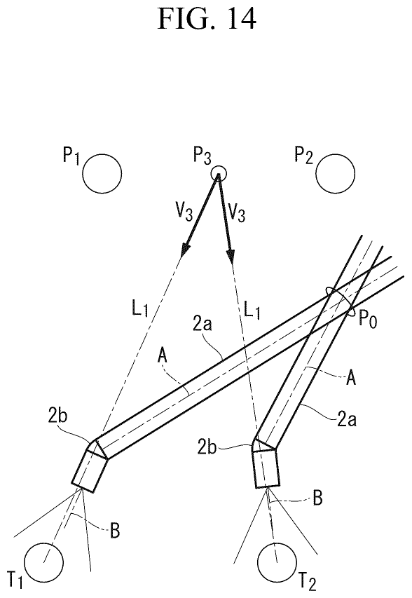

The same applies to the case where the endoscope 2 includes the bending portion 2b. In this case, as illustrated in FIG. 14, the processor 5a controls the moving device 3 and/or the bending portion 2b of the endoscope 2 so that the visual axis B is approximately parallel to the straight line L₁ and that the distance is smaller than a predetermined threshold. In this manner, the up-down direction of the endoscope image C acquired by the imaging element 2c can be easily made to coincide with the up-down direction recognized by the operator E without necessitating the process of rotating the endoscope image C. In this manner also, the operator E can intuitively operate the treatment tool 7.

Although the embodiments and modifications of the present disclosure have been described so far, the scope of the present disclosure is not limited by these, and various improvements are possible without departing from the gist of the present disclosure.

### REFERENCE SIGNS LIST

1 endoscope system
2 endoscope
2a scope barrel portion (insertion portion)
3 moving device
3a drive mechanism
5 control device
5a processor
5c storage unit (memory)
6 display device
7 treatment tool (first treatment tool, second treatment tool)
9 sensor
A longitudinal axis
B visual axis
C endoscope image (image)
C₄ plane
D patient (examination subject)
E operator
H head
P₀ camera port (third port)
P₁ first port
P₂ second port
T₁ focus target
V₁ first vector
V₂ second vector
V₃ third vector
α slope

1. An endoscope system comprising:
at least one processor comprising hardware;
an endoscope;
a holder that is configured to hold the endoscope and adjust a position and an orientation of the endoscope by a control signal from the at least one processor; and
a display that displays an image acquired by the endoscope,
wherein the at least one processor is configured to:
acquire a first position of a first port for inserting, into an examination subject, a first treatment tool operated by an operator, a second position of a second port for inserting, into the examination subject, a second treatment tool operated by the operator, and a third position of a focus target in the examination subject, and
rotate the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

2. The endoscope system according to claim 1, wherein the at least one processor is configured to define the first vector in a direction orthogonal to a plane that includes the first, second, and third positions.

3. The endoscope system according to claim 1, wherein the particular relative angle is 0°.

4. The endoscope system according to claim 1, wherein the particular relative angle is at least one of a preset value that has been set in advance or an offset value that has been input.

5. The endoscope system according to claim 1, wherein
the endoscope comprises a scope barrel that extends along a longitudinal axis direction thereof,
the holder comprises a drive joint that rotates the endoscope about the longitudinal axis, and
the at least one processor is configured to rotate the image by sending the control signal that actuates the drive joint.

6. The endoscope system according to claim 1, wherein the at least one processor is configured to rotate the image by image processing.

7. The endoscope system according to claim 2,
wherein the processor is further configured to:
calculate a third vector that connects the third position and a fourth position between the first position and the second position on the plane, and
in response to the third position changing, rotate the image by an angle calculated by a function of a change in angle of the third vector projected onto a horizontal plane.

8. The endoscope system according to claim 1, wherein
the endoscope comprises a scope barrel that extends along a longitudinal axis direction,
the endoscope system further comprises at least one memory,
the at least one memory is configured to store the first position, the second position, and a position of a third port into which the scope barrel is inserted, and
the at least one processor is configured to acquire, from the at least one memory, the first position, the second position, and the position of the third port.

9. The endoscope system according to claim 1, further comprising a sensor configured to detect a slope of a head of the operator,
wherein the at least one processor is configured to adjust a slope of the first vector on a basis of the slope detected by the sensor.

10. The endoscope system according to claim 1, wherein
the endoscope is of an oblique-viewing type or a type that comprises a bending portion in which a direction of the visual axis is changeable, and
the at least one processor is configured to control the holder and/or the endoscope so that the visual axis of the endoscope comes closer to a straight line that connects the third position and a fourth position between the first position and the second position.

11. A control device of an endoscope system comprising an endoscope, a holder that is configured to hold the endoscope and adjust a position and an orientation of the endoscope, and a display that displays an image acquired by the endoscope, the control device comprising:
at least one processor comprising hardware,
wherein the at least one processor is configured to:
acquire a first position of a first port for inserting, into an examination subject, a first treatment tool operated by an operator, a second position of a second port for inserting, into the examination subject, a second treatment tool operated by the operator, and a third position of a focus target in the examination subject, and
rotate the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

12. A control method of an endoscope system comprising an endoscope, a holder that is configured to hold the endoscope and adjust a position and an orientation of the endoscope, and a display that displays an image acquired by the endoscope, the control method comprising:
acquiring a first position of a first port for inserting, into an examination subject, a first treatment tool operated by an operator, a second position of a second port for inserting, into the examination subject, a second treatment tool operated by the operator, and a third position of a focus target in the examination subject; and
rotating the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.

13. A non-transitory computer-readable storage medium storing a program for controlling an endoscope system comprising an endoscope, a holder that is configured to hold the endoscope and adjust a position and an orientation of the endoscope, and a display that displays an image acquired by the endoscope, the program comprising instructing a computer to execute:
acquiring a first position of a first port for inserting, into an examination subject, a first treatment tool operated by an operator, a second position of a second port for inserting, into the examination subject, a second treatment tool operated by the operator, and a third position of a focus target in the examination subject; and
rotating the image on a basis of the acquired first, second, and third positions such that a particular relative angle is formed between a direction of a second vector, which is a projection of a first vector indicating an up-down direction of the focus target as viewed from the operator onto a plane orthogonal to a visual axis of the endoscope and an up-down direction of the image displayed on the display.
